(19) United States
(12) Patent Application Publication
(10) Pub. No.: US 2023/0343419 A1
(43) Pub. Date: Oct. 26, 2023

(54) METHOD, ELECTRONIC DEVICE, AND COMPUTER PROGRAM PRODUCT FOR MOLECULAR DOCKING

(71) Applicant: Dell Products L.P., Round Rock, TX (US)

(72) Inventors: Zijia Wang, WeiFang (CN); Sanping Li, Beijing (CN); Zhen Jia, Shanghai (CN)

(21) Appl. No.: 17/749,942

(22) Filed: May 20, 2022

(30) Foreign Application Priority Data
Apr. 22, 2022 (CN) 202210431202.6

(51) Int. Cl. G16C 20/50; G16C 20/30; G16C 20/70

(52) U.S. Cl. CPC G16C 20/50; G16C 20/30; G16C 20/70

(57) ABSTRACT

Embodiments of the present disclosure provide a method, an electronic device, and a computer program product for molecular docking. The method includes: determining a first feature representation characterizing a first molecule and a second feature representation characterizing a second molecule; determining a candidate region for the first molecule based at least on the first feature representation and the second feature representation, the candidate region comprising multiple candidate positions for docking the first molecule with the second molecule; and for each candidate position of the multiple candidate positions, determining a result of docking the first molecule with the second molecule at the candidate position. With the solution of the present disclosure, it is possible to calculate the docking result for the candidate region for the first molecule rather than the entire region, thereby reducing the amount of computation.

### RELATED APPLICATION(S)

The present application claims priority to Chinese Patent Application No. 202210431202.6, filed Apr. 22, 2022, and entitled “Method, Electronic Device, and Computer Program Product for Molecular Docking,” which is incorporated by reference herein in its entirety.

### FIELD

Embodiments of the present disclosure relate to the field of biological information, and in particular, to a method, an electronic device, and a computer program product for molecular docking.

### BACKGROUND

Molecular docking refers to theoretical simulation methods for determining the binding mode and affinity between molecules by studying interactions between molecules (e.g., ligands and receptors). Molecular docking may be applied to drug design, drug screening, compound generation, and other fields. Currently, software such as Dock, AutoDock, and FlexX have been proposed to implement molecular docking. However, due to complex spatial structures and physicochemical properties of molecules, a large quantity of computing resources is required to determine interactions between the molecules. Therefore, there is an urgent need for a method for molecular docking to efficiently determine intermolecular binding.

### SUMMARY

Embodiments of the present disclosure provide a solution for molecular docking.

In a first aspect of the present disclosure, a method for molecular docking is provided. The method includes: determining a first feature representation characterizing a first molecule and a second feature representation characterizing a second molecule; determining a candidate region for the first molecule based at least on the first feature representation and the second feature representation, the candidate region comprising multiple candidate positions for docking the first molecule with the second molecule; and for each candidate position of the multiple candidate positions, determining a result of docking the first molecule with the second molecule at the candidate position.

In a second aspect of the present disclosure, an electronic device is provided. The electronic device includes a processor and a memory coupled to the processor, the memory having instructions stored therein which, when executed by the processor, cause the device to execute actions. The actions include: determining a first feature representation characterizing a first molecule and a second feature representation characterizing a second molecule; determining a candidate region for the first molecule based at least on the first feature representation and the second feature representation, the candidate region comprising multiple candidate positions for docking the first molecule with the second molecule; and for each candidate position of the multiple candidate positions, determining a result of docking the first molecule with the second molecule at the candidate position.

In a third aspect of the present disclosure, a computer program product is provided. The computer program product is tangibly stored on a non-transitory computer-readable medium and includes machine-executable instructions. The machine-executable instructions, when executed by a machine, cause the machine to perform the method according to the first aspect.

In embodiments of the present disclosure, with the solution of molecular docking of the present disclosure, it is possible to calculate the docking result for the candidate region for the first molecule rather than the entire region, thereby reducing the amount of computation.

This Summary is provided to introduce the selection of concepts in a simplified form, which will be further described in the Detailed Description below. The Summary is neither intended to identify key features or main features of embodiments of the present disclosure, nor intended to limit the scope of the present disclosure.

### BRIEF DESCRIPTION OF THE DRAWINGS

The above and other objectives, features, and advantages of embodiments of the present disclosure will become more apparent from description provided herein of example embodiments of the present disclosure, in combination with the accompanying drawings. In the example embodiments of the present disclosure, the same reference numerals generally represent the same parts.

FIG. 1 illustrates a schematic diagram of an environment in which embodiments of the present disclosure can be implemented;

FIG. 2 illustrates an architectural diagram of a system for molecular docking according to some embodiments of the present disclosure;

FIG. 3 illustrates a flow chart of a method for molecular docking according to some embodiments of the present disclosure; and

FIG. 4 shows a block diagram of an example computing device that may be used to implement embodiments of the present disclosure.

### DETAILED DESCRIPTION

Principles of embodiments of the present disclosure will be described below with reference to several example embodiments shown in the accompanying drawings. Although example embodiments of the present disclosure are illustrated in the accompanying drawings, it should be understood that these embodiments are described only to enable those skilled in the art to better understand and then implement embodiments of the present disclosure, and not to limit the scope of the present disclosure in any way.

The term “include” and variants thereof used herein indicate open-ended inclusion, that is, “including but not limited to.” Unless specifically stated, the term “or” means “and/or.” The term “based on” means “based at least in part on.” The terms “an example embodiment” and “some embodiments” mean “at least one example embodiment.” The term “another embodiment” indicates “at least one additional embodiment.” The terms “first,” “second,” and the like may refer to different or identical objects. Other explicit and implicit definitions may also be included below.

As mentioned above, a number of solutions have been proposed for molecular docking. For example, AutoDock can lattice the three-dimensional structure of a molecule to be docked (i.e., receptor) and calculate a docking result (e.g., binding free energy) for each lattice point, thereby determining an optimal docking position based on the docking result for each lattice point. However, due to the number of lattice points being large, conventional molecular docking methods require a large amount of computing resources.

According to embodiments of the present disclosure, a solution for molecular docking is provided to solve at least one or more of the above problems or other potential problems. The solution includes: determining a first feature representation characterizing a first molecule and a second feature representation characterizing a second molecule; determining a candidate region for the first molecule based at least on the first feature representation and the second feature representation, the candidate region comprising multiple candidate positions for docking the first molecule with the second molecule; and for each candidate position of the multiple candidate positions, determining a result of docking the first molecule with the second molecule at the candidate position.

In this manner, the solution can determine the results of docking the first molecule and the second molecule for the multiple candidate positions in the candidate region without determining docking results for all lattice points in the first molecule. Thus, a large amount of computing resources can be saved.

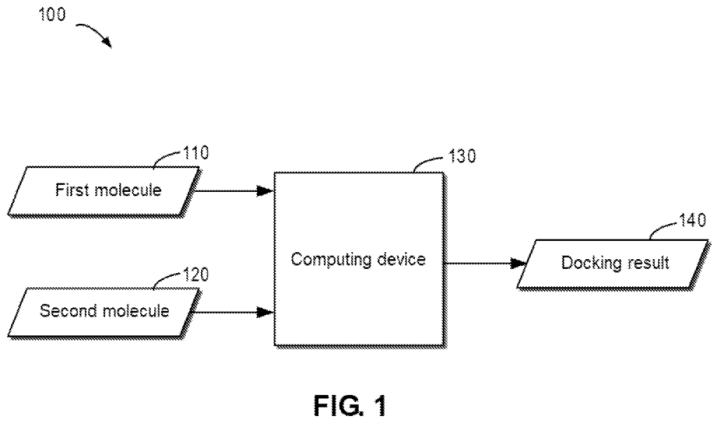

The basic principles and some example implementations of the present disclosure are illustrated below with reference to FIG. 1 to FIG. 4. It should be understood that these example embodiments are given only to enable those skilled in the art to better understand and thus implement embodiments of the present disclosure, and are not intended to limit the scope of the present disclosure in any way.

FIG. 1 shows example environment 100 in which embodiments of the present disclosure can be implemented. As shown in FIG. 1, environment 100 includes first molecule 110, second molecule 120, computing device 130, and docking result 140. First molecule 110 and second molecule 120 are two molecules to be docked. In some embodiments, first molecule 110 may be a receptor, and second molecule 120 may be a ligand. In some embodiments, first molecule 110 may be a larger molecule, and second molecule 120 may be a smaller molecule. In some embodiments, first molecule 110 may be a targeted protein, and second molecule 120 may be a candidate drug that serves as a ligand.

Computing device 130 includes a computing device in the form of a general-purpose computing device. In some implementations, computing device 130 may be implemented as a variety of user terminals or service terminals with computing capabilities. The service terminals may be servers provided by various service providers, large-scale computing devices, and the like. For example, the user terminals may be any type of mobile, fixed, or portable terminals, including a mobile phone, a site, a unit, a device, a multimedia computer, a multimedia tablet, an Internet node, a communicator, a desktop computer, a laptop computer, a notebook computer, a netbook computer, a tablet computer, a personal communication system (PCS) device, a personal navigation device, a personal digital assistant (PDA), an audio/video player, a digital camera/camcorder, a positioning device, a television receiver, a radio broadcast receiver, an e-book device, a gaming device, or any combination thereof, including accessories and peripherals of such devices, or any combination thereof.

Components of computing device 130 may include, but are not limited to, one or more processors or processing units, memories, storage devices, one or more communication units, one or more input devices, and one or more output devices. These components may be integrated on a single device or provided in the form of a cloud computing architecture. In the cloud computing architecture, these components may be remotely arranged and may work together to achieve the functions described in the present disclosure. In some implementations, cloud computing provides computing, software, data access, and storage services, which do not require terminal users to know physical locations or configurations of systems or hardware which provide these services. In various implementations, cloud computing provides services via a wide area network (e.g., the Internet) with appropriate protocols. For example, a cloud computing provider provides applications through a wide area network, and they are accessible through a web browser or any other computing components. Software or components of the cloud computing architecture and corresponding data may be stored on a server at a remote location. Computing resources in a cloud computing environment may be merged at a remote data center location, or they may be dispersed. Cloud computing infrastructures can provide services through a shared data center, even if they are each represented as a single access point for users. Therefore, the components and functions described herein may be provided from a service provider at a remote location by using the cloud computing architecture. Alternatively, they may also be provided from a conventional server, or they may be installed on a client terminal device directly or in other manners.

Computing device 130 may be used to implement the method for molecular docking according to embodiments of the present disclosure. As shown in FIG. 1, computing device 130 determines docking result 140 based on first molecule 110 and second molecule 120. Computing device 130 may receive, via its input device, information about first molecule 110 and second molecule 120 from other computing devices or storage devices.

The information may include amino acid sequences of first molecule 110 and second molecule 120. Each element in the amino acid sequences identifies a corresponding amino acid unit. For example, a molecule in an illustrative embodiment may be represented by the following amino acid sequence:

PIAQIHILEGRSDEQKETLIREVSEAISRSLDAPLTSVRVIITEMAKGHF
GIGGELASK

where each letter therein represents one type of amino acid unit.

Additionally, the information may include three-dimensional structures of first molecule 110 and second molecule 120. The three-dimensional structure may include coordinates of atoms that make up the molecule. Additionally, the information may include physicochemical properties of first molecule 110 and second molecule 120. The physicochemical property may include types of atoms, charge information, solubility parameter information, and the like.

Computing device 130 may determine docking result 140 based on the information about first molecule 110 and second molecule 120. Docking result 140 may include a binding strength (e.g., affinity, binding free energy, etc.) at at least one position (i.e., binding site) for docking first molecule 110 and second molecule 120. The at least one position may be ranked according to the binding strength. The at least one position may include the optimal position having the highest binding strength. Additionally, docking result 140 may include attitude information of first molecule 110 and second molecule 120 at the time of docking. The attitude information may include, for example, the orientation and conformation of the molecule.

It should be understood that environment 100 shown in FIG. 1 is merely an example and should not constitute any limitation to the functions and scope of the implementations described in the present disclosure. For example, computing device 130 may also acquire information about first molecule 110 and second molecule 120 from a storage device integrated therewith. Computing device 130 may also store determined docking result 140 in a storage device integrated therewith.

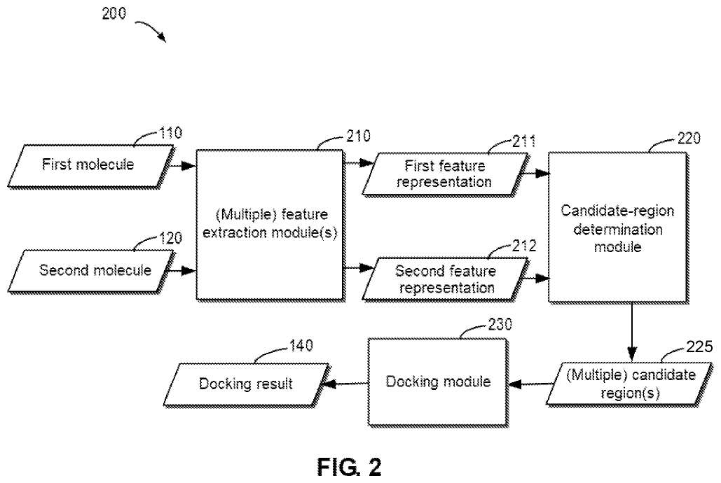

FIG. 2 illustrates an architectural diagram of system 200 for molecular docking according to embodiments of the present disclosure. System 200 may be implemented in computing device 130 shown in FIG. 1. As shown in FIG. 2, system 200 may include (multiple) feature extraction module(s) 210, candidate-region determination module 220, and docking module 230.

(Multiple) feature extraction module(s) 210 may determine first feature representation 211 characterizing first molecule 110 based on first molecule 110. (Multiple) feature extraction module(s) 210 may further determine second feature representation 212 characterizing second molecule 120 based on second molecule 120.

In some embodiments, (multiple) feature extraction module(s) 210 may be constructed based on any suitable machine learning method. For example, (multiple) feature extraction module(s) 210 may be part of an AlphaFold model.

The AlphaFold model is a model for predicting a three-dimensional structure based on the amino acid sequence of a protein. The AlphaFold model (including variants such as AlphaFold 2) primarily includes a feature extraction module, a structure prediction module, a function construction module, and a structure generation module. The details of the AlphaFold model are not repeated here.

In some embodiments, the feature extraction module in the AlphaFold model may be utilized as (multiple) feature extraction module(s) 210 for determining first feature representation 211 based on the amino acid sequence of first molecule 110 and determining second feature representation 212 based on the amino acid sequence of second molecule 120.

In some embodiments, the feature extraction module in the AlphaFold model may be further trained for determining first feature representation 211 and/or second feature representation 212 more accurately. Considering that the AlphaFold model is trained based on a generic protein data set, a specific training data set may be constructed based on first molecule 110 and/or second molecule 120 for further training of the AlphaFold model, so that the feature extraction module in the AlphaFold model can extract features more accurately for first molecule 110 and/or second molecule 120.

In some embodiments, (multiple) feature extraction module(s) 210 may include a first feature extraction module and a second feature extraction module, the first feature extraction module is further trained based on a training data set associated with first molecule 110, and the second feature extraction module is further trained based on a training data set associated with second molecule 120.

For example, in the case where second molecule 120 is a ligand, a training data set for the ligand may be constructed. By further training the AlphaFold model using this training data set, the feature extraction module may be enabled to better extract second feature representation 212 characterizing second molecule 120.

It should be understood that, when further training the feature extraction module in the AlphaFold model, it is possible to update parameters of the entire AlphaFold or to update only parameters of the feature extraction module while freezing parameters of other modules (such as the structure prediction module) in the AlphaFold model.

In some embodiments, (multiple) feature extraction module(s) 210 may acquire first feature representation 211 characterizing first molecule 110 and/or second feature representation 212 characterizing second molecule 120 from a database based on an identifier of first molecule 110. The database may store feature representations of various molecules that have been previously extracted.

Based on first feature representation 211 and second feature representation 212, candidate-region determination module 220 may determine (multiple) candidate region(s) 225 for first molecule 110, and each candidate region includes multiple candidate positions (i.e., candidate binding sites) for docking first molecule 110 and second molecule 120.

In some embodiments, the three-dimensional structure of first molecule 110 may be divided into multiple regions based on a variety of suitable approaches, and the scope of the present disclosure is not limited in this respect. The shape of the regions may be cuboid, cube, polyhedron, etc. Candidate-region determination module 220 may be constructed based on various suitable machine learning methods for determining (multiple) candidate region(s) 225 from the multiple regions. For example, candidate-region determination module 220 may be constructed as a multilayer perceptron (MLP), which includes multiple fully connected layers.

In some embodiments, candidate-region determination module 220 may form an end-to-end neural network model with (multiple) feature extraction module(s) 210. The neural network model may be trained based on a training data set. A sample in the training data set may include amino acid sequences of a pair of docked molecules and an identifier of the region in which the binding site of the pair of molecules is located.

In some embodiments, candidate-region determination module 220 may further determine (multiple) candidate region(s) 225 based on additional information. The additional information may include linking information about amino acid units of first molecule 110 and amino acid units of second molecule 120. The linking information may indicate which amino acid units in first molecule 110 may be linked to which amino acid units in second molecule 120. The linking information may include multiple pairs of amino acid units. In some embodiments, the linking information may be acquired based on expert knowledge.

In some embodiments, a third feature representation (not shown in FIG. 2) characterizing the linking between first molecule 110 and second molecule 120 may be determined by a third feature extraction module based on the linking information, and candidate-region determination module 220 may determine (multiple) candidate region(s) 225 based on first feature representation 211, second feature representation 212, and the third feature representation.

In some embodiments, the third feature extraction module may be obtained based on further training of the feature extraction module in the AlphaFold model. A training data set for the linking information may be constructed for use in training the AlphaFold model, thereby obtaining the third feature extraction module. A sample in the training data set for the linking information may include a pair of linked amino acid units and the coordinates of the center point of the three-dimensional structure of the pair of amino acid units.

In some embodiments, the feature representation characterizing the linking information may be extracted in other manners and input to candidate-region determination module 220 for determining (multiple) candidate region(s) 225.

Alternatively or additionally, the additional information may include attitude information of second molecule 120. The attitude information may include a priori knowledge indicative of the attitude of second molecule 120 when docked to first molecule 110. For example, the attitude information may indicate a common orientation of second molecule 120 when docked to other molecules similar to first molecule 110. In another example, the attitude information may indicate a preferred orientation of second molecule 120 based on expert knowledge.

Similarly, a feature representation characterizing the attitude information may be extracted in any suitable manner and input to candidate-region determination module 220 for determining (multiple) candidate region(s) 225.

Based on determined (multiple) candidate region(s) 225, docking module 230 may determine docking result 140 for each candidate position in (multiple) candidate region(s) 225. As described above, docking result 140 may include the binding strength at that candidate position for docking first molecule 110 and second molecule 120. Additionally, docking result 140 may include the attitude and/or conformation of first molecule 110 and/or second molecule 120 at the time of docking.

In some embodiments, conventional molecular docking methods may be utilized to determine docking result 140 at each candidate position. For example, determined (multiple) candidate region(s) 225 may be latticed using AutoDock, and the binding strength and/or affinity may be determined for each lattice point. Alternatively or additionally, a molecular docking method such as Dock or FlexX may be used to determine docking result 140 at each candidate position.

In some embodiments, a machine learning method may be used to determine the score of docking first molecule 110 and second molecule 120 at each candidate position in (multiple) candidate region(s) 225. The score may indicate docking result 140 of docking first molecule 110 and second molecule 120 at that candidate position. For example, the score may be determined using an empirical score function, a force field-based score function, and a knowledge-based score function.

Based on docking result 140, the candidate position with the highest binding strength may be determined as the optimal binding site for use in subsequent analysis. Additionally, although described here is the docking of second molecule 120 to first molecule 110, the docking results of first molecule 110 with multiple molecules may be determined to determine the molecule most easily docked to first molecule 110, thereby achieving drug screening.

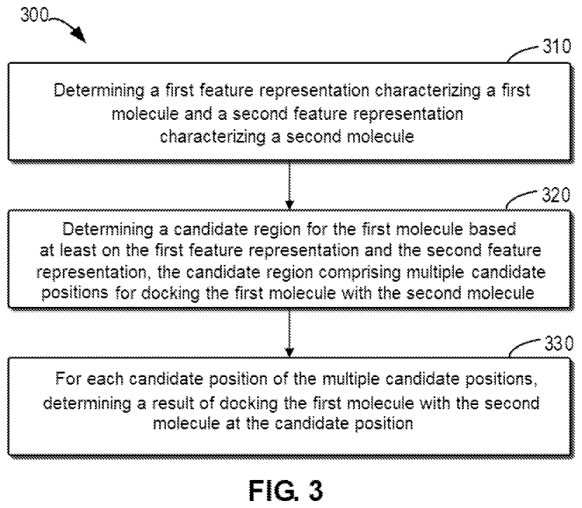

FIG. 3 illustrates a flow chart of example method 300 for molecular docking according to embodiments of the present disclosure. For example, method 300 may be performed by computing device 130 as shown in FIG. 1. It should be understood that method 300 may also include additional actions not shown and/or may omit actions shown, and the scope of the present disclosure is not limited in this regard. Method 300 is described in detail below with reference to FIG. 1.

At block 310, first feature representation 211 characterizing first molecule 110 and second feature representation 212 characterizing second molecule 120 are determined. In some embodiments, determining first feature representation 211 characterizing first molecule 110 and second feature representation 212 characterizing second molecule 120 includes: determining first feature representation 211 based on an amino acid sequence of first molecule 110; and determining second feature representation 212 based on an amino acid sequence of second molecule 120.

In some embodiments, determining first feature representation 211 characterizing first molecule 110 and second feature representation 212 characterizing second molecule 120 includes: determining the first feature representation and the second feature representation using at least one feature extraction module, wherein the at least one feature extraction module is part of an AlphaFold model.

In some embodiments, the at least one feature extraction module may include a first feature extraction module and a second feature extraction module, the first feature extraction module is further trained based on a training data set associated with first molecule 110, and the second feature extraction module is further trained based on a training data set associated with second molecule 120.

In some embodiments, first molecule 110 is a targeted protein and second molecule 120 is a ligand.

At block 320, candidate region 225 for first molecule 110 is determined based at least on first feature representation 211 and second feature representation 212, this candidate region 225 including multiple candidate positions for docking first molecule 110 with second molecule 120.

In some embodiments, determining candidate region 225 for first molecule 110 includes further determining candidate region 225 based on at least one of the following: linking information about amino acid units of first molecule 110 and amino acid units of second molecule 120; and attitude information of second molecule 120.

In some embodiments, determining candidate region 225 for first molecule 110 includes: determining candidate region 225 using a machine learning model.

At block 330, for each candidate position of the multiple candidate positions, a result of docking first molecule 110 with second molecule 120 at the candidate position is determined.

In some embodiments, determining a result of docking first molecule 110 with second molecule 120 at the candidate position includes: determining the result using a molecular docking algorithm, wherein the molecular docking algorithm comprises AutoDock.

In this manner, with embodiments according to the present disclosure, it is possible to reduce the amount of computation compared with conventional molecular docking methods, thereby efficiently determining a result of molecular docking. Therefore, embodiments according to the present disclosure can be implemented at an edge device with relatively few computing resources. For example, computing device 130 may be an edge device on the client terminal side.

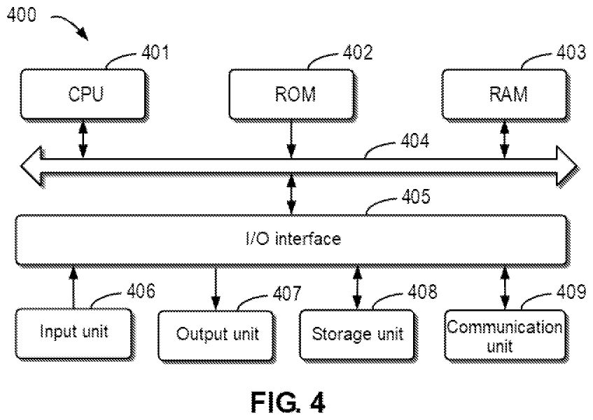

FIG. 4 is a schematic block diagram of example device 400 that can be used to implement embodiments of the present disclosure. For example, computing device 130 shown in FIG. 1 may be implemented by device 400. As shown in FIG. 4, device 400 includes central processing unit (CPU) 401 which may perform various appropriate actions and processing according to computer program instructions stored in read-only memory (ROM) 402 or computer program instructions loaded from storage unit 408 to random access memory (RAM) 403. RAM 403 may further store various programs and data required by operations of device 400. CPU 401, ROM 402, and RAM 403 are connected to each other through bus 404. Input/output (I/O) interface 405 is also connected to bus 404.

A plurality of components in device 400 are connected to I/O interface 405, including: input unit 406, such as a keyboard and a mouse; output unit 407, such as various types of displays and speakers; storage unit 408, such as a magnetic disk and an optical disc; and communication unit 409, such as a network card, a modem, and a wireless communication transceiver. Communication unit 409 allows device 400 to exchange information/data with other devices through a computer network such as the Internet and/or various telecommunication networks.

The various processes and processing described above, for example, method 300, may be performed by CPU 401. For example, in some embodiments, method 300 may be implemented as a computer software program that is tangibly included in a machine-readable medium such as storage unit 408. In some embodiments, part of or all the computer program may be loaded and/or installed onto device 400 via ROM 402 and/or communication unit 409. When the computer program is loaded into RAM 403 and executed by CPU 401, one or more actions of method 300 described above may be implemented.

Embodiments of the present disclosure include a method, an apparatus, a system, and/or a computer program product. The computer program product may include a computer-readable storage medium on which computer-readable program instructions for performing various aspects of the present disclosure are loaded.

The computer-readable storage medium may be a tangible device that may retain and store instructions used by an instruction-executing device. For example, the computer-readable storage medium may be, but is not limited to, an electric storage device, a magnetic storage device, an optical storage device, an electromagnetic storage device, a semiconductor storage device, or any suitable combination of the foregoing. More specific examples (a non-exhaustive list) of the computer-readable storage medium include: a portable computer disk, a hard disk, a RAM, a ROM, an erasable programmable read-only memory (EPROM or flash memory), a static random access memory (SRAM), a portable compact disc read-only memory (CD-ROM), a digital versatile disc (DVD), a memory stick, a floppy disk, a mechanical encoding device, for example, a punch card or a raised structure in a groove with instructions stored thereon, and any suitable combination of the foregoing. The computer-readable storage medium used herein is not to be interpreted as transient signals per se, such as radio waves or other freely propagating electromagnetic waves, electromagnetic waves propagating through waveguides or other transmission media (e.g., light pulses through fiber-optic cables), or electrical signals transmitted through electrical wires.

The computer-readable program instructions described herein may be downloaded from a computer-readable storage medium to various computing/processing devices or downloaded to an external computer or external storage device via a network, such as the Internet, a local area network, a wide area network, and/or a wireless network. The network may include copper transmission cables, fiber optic transmission, wireless transmission, routers, firewalls, switches, gateway computers, and/or edge servers. A network adapter card or network interface in each computing/processing device receives computer-readable program instructions from a network and forwards the computer-readable program instructions for storage in a computer-readable storage medium in the computing/processing device.

The computer program instructions for executing the operation of the present disclosure may be assembly instructions, instruction set architecture (ISA) instructions, machine instructions, machine-dependent instructions, microcode, firmware instructions, status setting data, or source code or object code written in any combination of one or more programming languages, the programming languages including object-oriented programming languages such as Smalltalk and C++, and conventional procedural programming languages such as the C language or similar programming languages. The computer-readable program instructions may be executed entirely on a user computer, partly on a user computer, as a stand-alone software package, partly on a user computer and partly on a remote computer, or entirely on a remote computer or a server. In a case where a remote computer is involved, the remote computer may be connected to a user computer through any kind of networks, including a local area network (LAN) or a wide area network (WAN), or may be connected to an external computer (for example, connected through the Internet using an Internet service provider). In some embodiments, an electronic circuit, such as a programmable logic circuit, a field programmable gate array (FPGA), or a programmable logic array (PLA), is customized by utilizing status information of the computer-readable program instructions. The electronic circuit may execute the computer-readable program instructions to implement various aspects of the present disclosure.

Various aspects of the present disclosure are described herein with reference to flow charts and/or block diagrams of the method, the apparatus (system), and the computer program product according to embodiments of the present disclosure. It should be understood that each block of the flow charts and/or the block diagrams and combinations of blocks in the flow charts and/or the block diagrams may be implemented by computer-readable program instructions.

These computer-readable program instructions may be provided to a processing unit of a general-purpose computer, a special-purpose computer, or a further programmable data processing apparatus, thereby producing a machine, such that these instructions, when executed by the processing unit of the computer or the further programmable data processing apparatus, produce means for implementing functions/actions specified in one or more blocks in the flow charts and/or block diagrams. These computer-readable program instructions may also be stored in a computer-readable storage medium, and these instructions cause a computer, a programmable data processing apparatus, and/or other devices to operate in a specific manner; and thus the computer-readable medium having instructions stored includes an article of manufacture that includes instructions that implement various aspects of the functions/actions specified in one or more blocks in the flow charts and/or block diagrams.

The computer-readable program instructions may also be loaded to a computer, a further programmable data processing apparatus, or a further device, so that a series of operating steps may be performed on the computer, the further programmable data processing apparatus, or the further device to produce a computer-implemented process, such that the instructions executed on the computer, the further programmable data processing apparatus, or the further device may implement the functions/actions specified in one or more blocks in the flow charts and/or block diagrams.

The flow charts and block diagrams in the drawings illustrate the architectures, functions, and operations of possible implementations of the systems, methods, and computer program products according to various embodiments of the present disclosure. In this regard, each block in the flow charts or block diagrams may represent a module, a program segment, or part of an instruction, the module, program segment, or part of an instruction including one or more executable instructions for implementing specified logical functions. In some alternative implementations, functions marked in the blocks may also occur in an order different from that marked in the accompanying drawings. For example, two successive blocks may actually be executed in parallel substantially, and sometimes they may also be executed in a reverse order, which depends on involved functions. It should be further noted that each block in the block diagrams and/or flow charts as well as a combination of blocks in the block diagrams and/or flow charts may be implemented by using a special hardware-based system that executes specified functions or actions, or implemented by using a combination of special hardware and computer instructions.

Example embodiments of the present disclosure have been described above. The above description is illustrative, rather than exhaustive, and is not limited to the disclosed various embodiments. Numerous modifications and alterations will be apparent to persons of ordinary skill in the art without departing from the scope and spirit of the illustrated embodiments. The selection of terms used herein is intended to best explain the principles and practical applications of the various embodiments or the improvements to technologies on the market, so as to enable persons of ordinary skill in the art to understand the embodiments disclosed herein.

What is claimed is:

1. A method for molecular docking, comprising:
determining a first feature representation characterizing a first molecule and a second feature representation characterizing a second molecule;
determining a candidate region for the first molecule based at least on the first feature representation and the second feature representation, the candidate region comprising multiple candidate positions for docking the first molecule with the second molecule; and
for each candidate position of the multiple candidate positions, determining a result of docking the first molecule with the second molecule at the candidate position.

2. The method according to claim 1, wherein determining a first feature representation characterizing a first molecule and a second feature representation characterizing a second molecule comprises:
determining the first feature representation based on an amino acid sequence of the first molecule; and
determining the second feature representation based on an amino acid sequence of the second molecule.

3. The method according to claim 1, wherein determining a first feature representation characterizing a first molecule and a second feature representation characterizing a second molecule comprises:
determining the first feature representation and the second feature representation using at least one feature extraction module, wherein the at least one feature extraction module is part of an AlphaFold model.

4. The method according to claim 3, wherein the at least one feature extraction module comprises a first feature extraction module and a second feature extraction module, the first feature extraction module is further trained based on a training data set associated with the first molecule, and the second feature extraction module is further trained based on a training data set associated with the second molecule.

5. The method according to claim 1, wherein determining the candidate region for the first molecule comprises further determining the candidate region based on at least one of the following:
linking information about amino acid units of the first molecule and amino acid units of the second molecule; and
attitude information of the second molecule.

6. The method according to claim 1, wherein determining a candidate region for the first molecule comprises:
determining the candidate region using a machine learning model.

7. The method according to claim 1, wherein determining a result of docking the first molecule with the second molecule at the candidate position comprises:
determining the result using a molecular docking algorithm, wherein the molecular docking algorithm comprises AutoDock.

8. The method according to claim 1, wherein the first molecule is a targeted protein, and the second molecule is a ligand.

9. An electronic device, comprising:
a processor; and
a memory coupled to the processor, wherein the memory has instructions stored therein, and the instructions, when executed by the processor, cause the device to execute actions comprising:
determining a first feature representation characterizing a first molecule and a second feature representation characterizing a second molecule;
determining a candidate region for the first molecule based at least on the first feature representation and the second feature representation, the candidate region comprising multiple candidate positions for docking the first molecule with the second molecule; and
for each candidate position of the multiple candidate positions, determining a result of docking the first molecule with the second molecule at the candidate position.

10. The device according to claim 9, wherein determining a first feature representation characterizing a first molecule and a second feature representation characterizing a second molecule comprises:
determining the first feature representation based on an amino acid sequence of the first molecule; and
determining the second feature representation based on an amino acid sequence of the second molecule.

11. The device according to claim 9, wherein determining a first feature representation characterizing a first molecule and a second feature representation characterizing a second molecule comprises:
determining the first feature representation and the second feature representation using at least one feature extraction module, wherein the at least one feature extraction module is part of an AlphaFold model.

12. The device according to claim 11, wherein the at least one feature extraction module comprises a first feature extraction module and a second feature extraction module, the first feature extraction module is further trained based on a training data set associated with the first molecule, and the second feature extraction module is further trained based on a training data set associated with the second molecule.

13. The device according to claim 9, wherein determining the candidate region for the first molecule comprises further determining the candidate region based on at least one of the following:
linking information about amino acid units of the first molecule and amino acid units of the second molecule; and
attitude information of the second molecule.

14. The device according to claim 9, wherein determining a candidate region for the first molecule comprises:
determining the candidate region using a machine learning model.

15. The device according to claim 9, wherein determining a result of docking the first molecule with the second molecule at the candidate position comprises:
determining the result using a molecular docking algorithm, wherein the molecular docking algorithm comprises AutoDock.

16. The device according to claim 9, wherein the first molecule is a targeted protein, and the second molecule is a ligand.

17. A computer program product tangibly stored on a non-transitory computer-readable medium and comprising machine-executable instructions, wherein the machine-executable instructions, when executed by a machine, cause the machine to perform a method for molecular docking, the method comprising:
determining a first feature representation characterizing a first molecule and a second feature representation characterizing a second molecule;
determining a candidate region for the first molecule based at least on the first feature representation and the second feature representation, the candidate region comprising multiple candidate positions for docking the first molecule with the second molecule; and
for each candidate position of the multiple candidate positions, determining a result of docking the first molecule with the second molecule at the candidate position.

18. The computer program product according to claim 17, wherein determining a first feature representation characterizing a first molecule and a second feature representation characterizing a second molecule comprises:
determining the first feature representation based on an amino acid sequence of the first molecule; and
determining the second feature representation based on an amino acid sequence of the second molecule.

19. The computer program product according to claim 17, wherein determining a first feature representation characterizing a first molecule and a second feature representation characterizing a second molecule comprises:
determining the first feature representation and the second feature representation using at least one feature extraction module, wherein the at least one feature extraction module is part of an AlphaFold model.

20. The computer program product according to claim 19, wherein the at least one feature extraction module comprises a first feature extraction module and a second feature extraction module, the first feature extraction module is further trained based on a training data set associated with the first molecule, and the second feature extraction module is further trained based on a training data set associated with the second molecule.
